(19) United States
(12) Patent Application Publication
(10) Pub. No.: US 2007/0049757 A1
(43) Pub. Date: Mar. 1, 2007

(54) Methods for the synthesis of substituted amino uracils

(76) Inventors: Jayson M. Rieger, Charlottesville, VA (US); Robert Thompson, Charlottesville, VA (US)

Correspondence Address: PROSKAUER ROSE LLP, 1001 PENNSYLVANIA AVE, N.W.,, SUITE 400 SOUTH, WASHINGTON, DC, 20004, US

(21) Appl. No.: 11/510,632

(22) Filed: Aug. 28, 2006

Related U.S. Application Data
(60) Provisional application No. 60/711,422, filed on Aug. 26, 2005.

(51) Int. Cl. C07D 239/46; C07D 403/02

(52) U.S. Cl. 544/309; 544/310

(57) ABSTRACT

The invention provides novel methods of preparing substituted amino uracil compounds that can be employed as useful intermediates in the synthesis of various xanthines. Highly regioselective unsymmetrical amino uracils can be obtained using these methods which is a particular advantage over other existing methods.

This application claims priority to Application Ser. No. 60/711,422, filed Aug. 26, 2005, the contents of which are hereby incorporated by reference in their entirety.

### BACKGROUND OF THE INVENTION

Substituted amino uracils are useful intermediates in the preparation of a variety of xanthines. It has been reported that therapeutic concentrations of xanthines such as theophylline or enprofylline block human A_{2B} receptors, and it has been proposed that antagonists selective for this subtype may have potential use as antiasthmatic agents. (See Feoktistov, I., et al., Pharmacol. Rev. 1997, 49, 381-402; and Robeva, A. S., et al., Drug Dev. Res. 1996, 39, 243-252). Enprofylline has a reported Kᵢ value of 7 μM and is somewhat selective in binding to human A_{2B} adenosine receptors. (See Robeva, A. S., et al., Drug Dev. Res. 1996, 39, 243-252 and Linden, J., et al., Mol. Pharmacol. 1999, 56, 705-713).

A_{2B} adenosine receptors are expressed in some mast cells, such as the BR line of canine mastocytoma cells, which appear to be responsible for triggering acute Ca²⁺ mobilization and degranulation. (See Auchampach, J. A., et al., Mol. Pharmacol. 1997, 52, 846-860 and Forsyth, P., et al., Inflamm. Res. 1999, 48, 301-307). A_{2B} adenosine receptors also trigger Ca²⁺ mobilization, and participate in a delayed release of IL8 from human HMC-1 mast cells. Other functions associated with the A_{2B} adenosine receptors are the control of cell growth and gene expression, (See Neary, J., et al., Trends Neurosci. 1996, 19, 13-18) endothelial-dependent vasodilation (See Martin, P. L., et al., J. Pharmacol. Exp. Ther. 1993, 265, 248-253), and fluid secretion from intestinal epithelia. (See Strohmeier, G. R., et al., J Biol. Chem. 1995, 270, 2387-2394). Adenosine acting through A_{2B} Adenosine receptors has also been reported to stimulate chloride permeability in cells expressing the cystic fibrosis transport regulator. (See Clancy, J. P., et al., Am. J Physiol. 1999, 276, C361-C369).

A number of substituted amino uracils have been shown to have diuretic activity. Experimental studies on animals have shown that the diuretic activity of several substituted amino uracils was equal to that shown by xanthines such as caffeine, theophylline and theobromine. At the same time their toxicity was found to be considerably lower. (See Papesch, V. et al., J. Org. Chem. 1951, 16, 1879-1890).

Traditional methods for the synthesis of substituted amino uracils involve treatment of alkyl substituted ureas with reagents such as acetic anhydride and cyanoacetic acid at elevated temperatures followed by cyclization with lithium hydroxide. The disadvantages of these methods include the inability to control the regioselectivity of the reaction. As a result, mixtures of two regio-isomeric products are obtained and the isolation and purification is complicated by the need for cumbersome chromatographic techniques. This lack of regioselectivity also results in diminished yields of the specific isomer desired.

Due to the importance of substituted amino uracils not only as diuretics but also as intermediates in the preparation of xanthine based adenosine receptor antagonists, there exists a need for improved processes to synthesize these compounds. More particularly, there is a need for new methods that allow complete control of the regioselectivity of the products obtained.

### SUMMARY OF THE INVENTION

It is therefore an object of the invention to provide novel methods of preparing substituted amino uracil compounds.

In is another object of the present invention to provide novel intermediates useful for preparing substituted amino uracil compounds.

The present invention is intended to encompass all pharmaceutically acceptable ionized forms (e.g., salts) and solvates (e.g., hydrates) of the compounds, regardless of whether such forms and solvates are specified, as it is well known in the art that pharmaceutical agents in an ionized or solvated form may be used. Unless a particular stereochemistry is specified, recitation of a compound is intended to encompass all possible stereoisomers (e.g., enantiomers or diastereomers), independent of whether the compound is present as an individual isomer or a mixture of isomers. A recitation of a compound is intended to include all possible resonance forms and tautomers. Claims to the compound of the present invention are intended to encompass the compound and all pharmaceutically acceptable ionized forms and solvates, all possible stereoisomers, resonance forms and tautomers, unless otherwise specifically specified.

Other objects, features, and advantages of the present invention will become apparent from the following detailed description. It should be understood, however, that the detailed description and the specific examples, while indicating preferred embodiments of the present invention, are given by way of illustration only, since various changes and modifications within the spirit and scope of the invention will become apparent to those skilled in the art from the detailed description.

### DETAILED DESCRIPTION OF THE INVENTION

The present invention provides novel methods of preparing substituted amino uracil compounds that are useful intermediates in the synthesis of various xanthines. Unsymmetrical amino uracils can be obtained in a highly regioselective manner using these methods, which provides a particular advantage over existing methods.

In an embodiment, the present invention provides a process for preparing a compound of formula I or a pharmaceutically acceptable salt thereof, comprising: cyclizing a compound of formula II in the presence of a strong base:

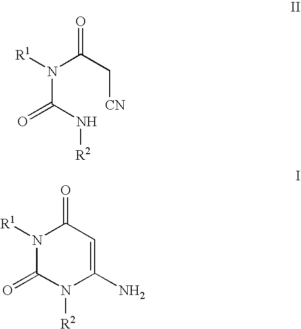

wherein:

R¹ and R² are independently hydrogen or selected from the group consisting of (C₁₋₈)alkyl, halo(C₁₋₈)alkyl, (C₃₋₈)alkenyl, (C₃₋₈)alkenyl(C₁₋₈)alkyl-, (C₃₋₈)alkynyl, (C₃₋₈) alkynyl(C₁₋₈)alkyl-,(C₁₋₈)alkoxy, (C₃₋₈)cycloalkyl, (C₃₋₈)cycloalkyl(C₁₋₈)alkyl-, (C₄₋₁₀) heterocyclyl, (C₄₋₁₀)heterocyclyl(C₁₋₈)alkyl-, (C₆₋₁₀)aryl, (C₆₋₁₀)aryloxy, (C₆₋₁₀)aryl(C₁₋₈) alkyl-, (C₅₋₁₀)heteroaryl, and (C₅₋₁₀)heteroaryl(C₁₋₈)alkyl-, wherein each group is independently substituted or unsubstituted.

In another embodiment, the present invention provides a process for preparing a compound of formula II, comprising:

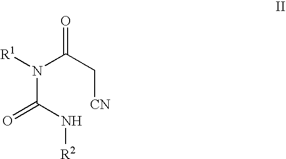

a) contacting a compound of formula III with an acylating agent of formula IV in the presence of a second strong base to produce a compound of formula V; and,

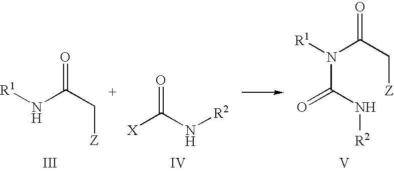

b) (i) when Z is a leaving group, contacting the compound of Formula V with M-CN to produce a compound of formula II; or,

(ii) when Z is a group that can be converted to a leaving group, converting the group to a leaving group and contacting the resulting compound with M-CN to produce a compound of formula II;

R¹ and R² are independently hydrogen or selected from the group consisting of (C₁₋₈) alkyl, halo(C₁₋₈)alkyl, (C₃₋₈)alkenyl, (C₃₋₈)alkenyl(C₁₋₈)alkyl-, (C₃₋₈)alkynyl, (C₃₋₈) alkynyl(C₁₋₈)alkyl-, (C₁₋₈)alkoxy, (C₃₋₈)cycloalkyl, (C₃₋₈)cycloalkyl(C₁₋₈)alkyl-, (C₄₋₁₀)heterocyclyl, (C_{4 -10})heterocyclyl(C₁₋₈)alkyl-, (C₆₋₁₀)aryl, (C₆₋₁₀)aryloxy, (C₆₋₁o)aryl(C₁₋₈)alkyl-, (C₅₋₁₀)heteroaryl, and (C₅₋₁₀)heteroaryl(C₁₋₈)alkyl-, wherein each group is independently substituted or unsubstituted;

M is Na or K;

Z is selected from the group consisting of CN, a leaving group, or a group that can be converted to a leaving group; and,

X is a leaving group.

In another embodiment, the present invention provides a process for preparing a compound of formula II, comprising:

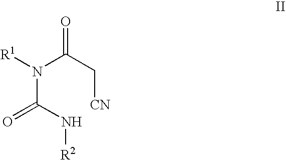

a) contacting a compound of formula III with an acylating agent of the formula VI in the presence of a third strong base to produce a compound of formula VII:

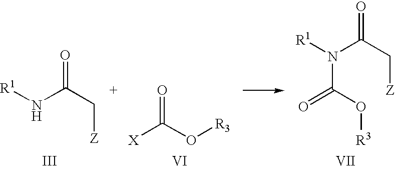

a-1) contacting the compound of formula VII with an amine of the formula R²-NH₂ to produce a compound of formula V:

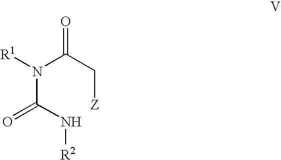

b) (i) when Z is a leaving group, contacting the compound of Formula V with M-CN to produce a compound of formula II; or,

(ii) when Z is a group that can be converted to a leaving group, converting the group to a leaving group and contacting the resulting compound with M-CN to produce a compound of formula II;

R¹ and R2 are independently hydrogen or selected from the group consisting of (C₁₋₈)alkyl, halo(C₁₋₈)alkyl, (C₃₋₈)alkenyl, (C₃₋₈)alkenyl(C₁₋₈)alkyl-, (C₃₋₈)alkynyl, (C₃₋₈)alkynyl(C₁₋₈)alkyl-, (C₁₋₈)alkoxy, (C₃₋₈)cycloalkyl, (C₃₋₈)cycloalkyl(C₁₋₈)alkyl-, (C₄₋₁₀)heterocyclyl, (C₄₋₁₀)heterocyclyl(C₁₋₈)alkyl-, (C₆₋₁₀)aryl, (C₆₋₁₀)aryloxy, (C₆₋₁₀)aryl(C₁₋₈)alkyl-, (C₅₋₁₀)heteroaryl, and (C₋₁₀)heteroaryl(C₁₋₈)alkyl-, wherein each group is independently substituted or unsubstituted;

OR³ is a leaving group;

M is Na or K;

Z is selected from the group consisting of CN, a leaving group, or a group that can be converted to a leaving group; and,

X is a leaving group.

In another embodiment, the present invention provides a process, further comprising:

c) cyclizing the compound of formula II in the presence of a fourth strong base to form a compound of formula I or a pharmaceutically acceptable salt thereof:

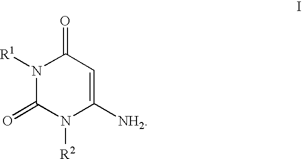

In another embodiment, the present invention provides a process wherein: the strong base is a metal hydroxide.

In another embodiment, the present invention provides a process wherein the metal hydroxide base is selected from the group consisting of lithium hydroxide, calcium hydroxide, sodium hydroxide, potassium hydroxide, and mixtures thereof.

In another embodiment, the present invention provides a process wherein: the metal hydroxide base is sodium hydroxide or lithium hydroxide.

In another embodiment, the present invention provides a process wherein the strong base is selected from a group consisting of n-butyl lithium, LDA, LiHMDS, NaHMDS, and KHMDS.

In another embodiment, the present invention provides a process wherein the base is NaHMDS or KHMDS.

In another embodiment, the present invention provides a process wherein:

R¹ is selected from a group consisting of (C₃₋₅)cycloalkyl, (C₃₋₅)cycloalkyl(C₁₋₃)alkyl-, (C₁₋₅)alkyl, and aryl(C₁₋₃)alkyl-;

R² is selected from a group consisting of (C₃₋₅)cycloalkyl, (C₃₋₅)cycloalkyl(C₁₋₃)alkyl-, (C₁₋₅)alkyl, and aryl(C₁₋₃)alkyl-; and,

X is selected from a group consisting of Cl, Br, and I.

In another embodiment, the present invention provides a process wherein:

R¹ is selected from a group consisting of ethyl, propyl, cyclopropyl, cyclobutyl, cyclopropylmethylene, cyclobutylmethylene, and benzyl;

R² is selected from a group consisting of ethyl, propyl, cyclopropyl, cyclobutyl, cyclopropylmethylene, cyclobutylmethylene, and benzyl; and,

X is selected from a group consisting of Cl, Br, and I.

In another embodiment, the present invention provides a process wherein:

R¹ is selected from a group consisting of (C₃₋₅)cycloalkyl, (C₃₋₅)cycloalkyl(C₁₋₃)alkyl-, (C₁₋₅)alkyl, and aryl(C₁₋₃)alkyl-;

R² is selected from a group consisting of (C₃₋₅)cycloalkyl, (C₃₋₅)cycloalkyl(C₁₋₃)alkyl-, (C₁₋₅)alkyl, and aryl(C₁₋₃)alkyl-;

R³ is selected from a group consisting of (C₁₋₈)alkyl, aryl(C₁₋₃)alkyl-, and aryl; and,

X is selected from a group consisting of Cl, Br, and I.

In another embodiment, the present invention provides a process wherein:

R¹ is selected from a group consisting of ethyl, propyl, cyclopropyl, cyclobutyl, cyclopropylmethylene, cyclobutylmethylene, and benzyl;

R² is selected from a group consisting of ethyl, propyl, cyclopropyl, cyclobutyl, cyclopropylmethylene, cyclobutylmethylene, and benzyl;

R³ is selected from a group consisting of methyl, ethyl, phenyl, and benzyl; and,

X is selected from a group consisting of Cl, Br, and I.

In another embodiment, the present invention provides a process wherein R¹ is cyclopropyl and R2 is propyl.

The following definitions are used, unless otherwise described. The examples given for each definition are non-limiting, unless otherwise indicated.

“Halo” means fluoro, chloro, bromo, or iodo.

“Alkyl”, “alkoxy”, “alkenyl”, “alkynyl”, etc. denote both straight and branched groups; but reference to an individual radical such as “propyl” or “isopropyl” refer only to the straight or branched groups respectively. “C_{x-y}alkyl” are used where X and Y indicate the number of carbon atoms in the chain. For example, C₁₋₄alkyl include alkyl groups that have a chain between 1 and 4 carbon atoms (e.g., methyl, ethyl, propyl, isopropyl, butyl, sec-butyl, isobutyl, etc.). When alkyl can be partially unsaturated, the alkyl chain may comprise one or more (e.g., 1, 2, 3, or 4) double or triple bonds in the chain.

“Aryl” denotes a phenyl radical or an ortho-fused bicyclic carbocyclic radical having about nine to ten ring atoms in which at least one ring is aromatic.

“Arylalkyl” or “(C₆₋₁₀)aryl(C₁₋₈)alkyl-” refer to a group of the formula aryl(C₁₋₈)alkyl-, where aryl and (C₁₋₈)alkyl are as defined herein.

“Heterocycle” encompasses a cyclic radical attached or linked via a nitrogen or carbon ring atom of a monocyclic, fused-bicyclic, or bridged-bicyclic, saturated or unsaturated, ring system containing 5-10 ring atoms and preferably from 5-6 ring atoms, consisting of carbon and one, two, three or four hetero atoms including, for example, non-peroxide oxy (—O—), thio (—S—), sulfinyl (—SO—), sulfonyl (—S(O)₂—), amine —N(R)—, —N(O)—, —N═, phosphorus (—P—), —P(O)— and the like, where the group R is as defined herein, and optionally containing 1-3 double bonds (e.g., —CH═CH— or —CH═N—). Fully unsaturated heterocycles may also be defined as “heteroaryls.” Heterocycle includes, for example, tetrahydrofuryl, dihydrofuryl, tetrahydroimidazolyl, azanorbomyl, pyrrolidyl, piperidyl, piperizinyl, morpholinyl, azepinyl, 1,3-diazepinyl, 1,3-benzodiazepinyl, 1,4-diazepinyl, 1,4-benzodiazepinyl, 1,5-diazepinyl, 1,5-benzodiazepino and the like.

“Heteroaryl” encompasses a radical attached via a ring atom of a monocyclic or bicyclic aromatic ring containing 5-14 ring atoms, such as a monocyclic containing from 5-6 ring atoms, comprising carbon and one, two, three or four hetero atoms including, for example, non-peroxide oxy (—O—), thio (—S—), sulfinyl (—SO—), sulfonyl (—S(O)₂—), amine —N(R)—, —N(O)—, —N═ and the like, where the group R is as defined herein. Bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, pteridine, purine, carbazole, acridine and the like. Preferred heteroaryl groups include imidazolyl, triazolyl, triazinyl, oxazoyl, isoxazoyl, thiazolyl, isothiazoyl, thiodiazolyl, pyrrolyl, pyrazolyl, pyrazinyl, tetrazolyl, pyridinyl, pyrimidinyl, indolyl, isoquinolyl, quinolyl and the like.

Specifically, (C₁₋₈)alkyl can be methyl, ethyl, n-propyl, isopropyl, n-butyl, iso-butyl, sec-butyl, tert-butyl, n-pentyl, isopentyl, 3-pentyl, n-hexyl, n-heptyl, n-octyl or the branched (C₃₋₈)alkyl; (C₂₋₈)alkenyl can be vinyl, 1-propenyl, 2-propenyl (allyl), 1-butenyl, 2-butenyl, 3-butenyl, 1-pentenyl, 2-pentenyl, 3-pentenyl, 1-hexenyl, 2-hexenyl, 3-hexenyl, 1-heptenyl, 2-heptenyl, 3-heptenyl, l-octenyl, 2-octenyl, 3-octenyl, 4-octenyl or the branched (C₃₋₈)alkenyl; (C₃₋₈)alkenyl can be 2-propenyl (allyl), 2-butenyl, 3-butenyl, 2-pentenyl, 3-pentenyl, 1-hexenyl, 2-hexenyl, 3-hexenyl, 2-heptenyl, 3-heptenyl, 2-octenyl, 3-octenyl, 4-octenyl, or the branched (C₃₋₈)alkenyl; (C₂₋₈)alkynyl can be ethynyl, 1-propynyl, 2-propynyl (propargyl), 1 -butynyl, 2-butynyl, 3-butynyl, 1-pentynyl, 2-pentynyl, 3-pentynyl, 1-hexynyl, 2-hexynyl, 3-hexynyl, 1-heptynyl, 2-heptynyl, 3-heptynyl, 1-octynyl, 2-octynyl, 3-octynyl, 4-octynyl, or the branched (C₃₋₈)alkynyl; (C₃₋₈)alkynyl can be 2-propynyl (propargyl), 2-butynyl, 3-butynyl, 1-pentynyl, 2-pentynyl, 3-pentynyl, 1-hexynyl, 2-hexynyl, 3-hexynyl, 1-heptynyl, 2-heptynyl, 3-heptynyl, 1-octynyl, 2-octynyl, 3-octynyl, 4-octynyl, or the branched (C₃₋₈)alkynyl; (C₁₋₈)alkoxy can be methoxy, ethoxy, n-propoxy, isopropoxy, n-butoxy, iso-butoxy, sec-butoxy, tert-butoxy, pentoxy, 3-pentoxy, n-hexyloxy, n-heptyloxy, n-octyloxy, or the branched (C₃₋₈)alkoxy; halo(C₁₋₈)alkyl can be iodomethyl, bromomethyl, chloromethyl, fluoromethyl, trifluoromethyl, 2-chloroethyl, 2-bromoethyl, 2-fluoroethyl, 3-fluoropropyl, 2,2,2-trifluoroethyl, pentafluoroethyl, or the branched halo(C₃₋₈)alkyl; (C₃₋₈)cycloalkyl can be cyclopropyl, cyclobutyl, cyclopentyl, cyclohexyl, cycloheptyl or cyclooctyl; (C₃₋₈)cycloalkyl(C₁₋₈)alkyl- can be cyclopropylmethyl, cyclobutylmethyl, cyclopentylmethyl, cyclohexylmethyl, 2-cyclopropylethyl, 2-cyclobutylethyl, 2-cyclopentylethyl or 2-cyclohexylethyl; (C₆₋₁₀)aryl can be phenyl, indenyl or naphthyl.

Arylalkyl includes phenylethyl, benzyl, 2-phenylpropyl, 3-phenylpropyl, 2-naphthylmethyl or 3-naphthylmethyl; and heteroaryl can be, for example, imidazolyl, triazolyl, triazinyl, oxazoyl, isoxazoyl, thiazolyl, isothiazoyl, pyrrolyl, pyrazinyl, tetrazolyl, pyridyl, pyrimidinyl, indolyl, isoquinolyl, quinolyl, or an oxide thereof.

Specific cycloalkyl groups include cyclopropyl, cyclobutyl, cyclopentyl and cyclohexyl.

Specific cycloalkylalkyl- groups include cyclopropylmethyl, cyclobutylmethyl, cyclopropylethyl, cyclopentylmethyl, cyclohexylmethyl, 2-cyclopentylethyl, and 2-cyclohexylethyl.

“Pharmaceutically acceptable salts” means salts of the compounds of the present invention which are pharmaceutically acceptable and which have the desired pharmacological activity. Such salts include acid addition salts formed with inorganic acids such as hydrochloric acid, hydrobromic acid, sulfuric acid, nitric acid, phosphoric acid, and the like. The salt may also be formed with organic acids including acetic acid, propionic acid, hexanoic acid, heptanoic acid, glycolic acid, pyruvic acid, lactic acid, malonic acid, succinic acid, malic acid, maleic acid, flimaric acid, tartatic acid, citric acid, benzoic acid, gluconic acid, glutamic acid, and the like.

A “leaving group” as used herein, is a moiety that may be displaced in a chemical transformation, such as a nucleophilic displacement reaction, an acylation reaction and the like; and non-exclusive examples of such moiety include hydrogen, hydroxyl, halides, triflates, mesylate, tosylate, acetate, triazolyl, imidazolyl or cyano, and the like. The present Z leaving group is displaceable by M-CN. The present X leaving group is displaceable by the amide nitrogen (e.g., deprotonated) of formula III. The OR³ leaving group is displaceable by the amine nitrogen of R²NH₂.

A typical sequence of reactions to obtain the target unsymmetrical substituted amino uracils is shown in scheme 1. As depicted in scheme 1, a variety of alkyl amines can be reacted with activated carbonyls such as acid chlorides or acid anhydrides under standard basic conditions to obtain the corresponding amides. The reaction can be carried out using neat reagents or employing dry ethereal solvents such as diethyl ether or THF under an inert atmosphere. These amides can be further reacted with another amide containing an activated carbonyl under strong basic conditions at low temperatures to directly obtain the corresponding diamide. Alternatively, a two step process can be employed wherein in the first step, the amide is reacted with an alkoxy acid halide or a mixed anhydride under strongly basic conditions and sub-zero temperatures to obtain a carbamate-like intermediate which in turn can be reacted with a second alkyl amine to afford the corresponding diamide. It may be necessary to cool the reaction vessel to temperatures as low as −78° C., although typically a range of−35-60° C. is maintained. The reactions are carried out under an inert atmosphere to obtain a higher yield of the product. Again, dry ethereal solvents are typically used for this reaction step. The reaction of the carbamate-like intermediate with the second amine can be heated to speed up the reaction or if the reaction is too sluggish. Typically, the reaction is carried out in the range of 40-80° C.

The Z group can be either CN, or a leaving group including but not limited to Cl, Br, I, or Z can be a group that can be converted to a leaving group (e.g., OH). When the Z group is other than a cyano, it can be converted into a cyano group by a standard activation and displacement sequence. A typical scenario is when the Z group is an OH group. Activation can be achieved by converting the free hydroxy group to a leaving group. Suitable leaving groups include but are not limited to, the corresponding triflate, mesylate or tosylate. Displacement of the activated hydroxy group can be accomplished using a cyanide salt such as NaCN or KCN. Alternatively, if the Z group is a halide or other leaving group, a direct displacement with NaCN or KCN affords the corresponding nitrile.

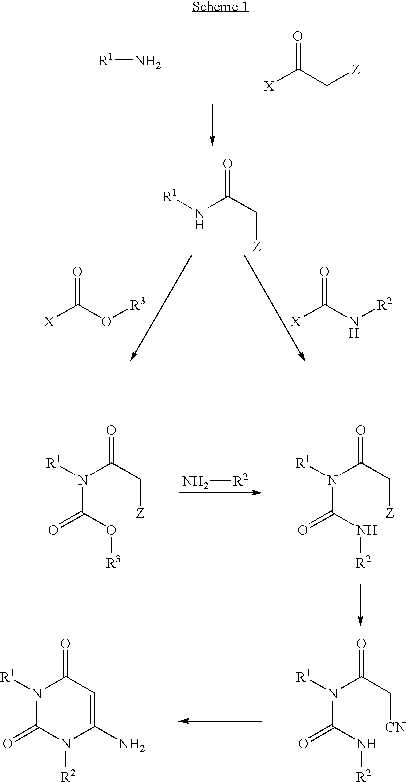

Once the corresponding nitrile is isolated, the final cyclization can be conducted under basic conditions. A variety of bases such as aqueous solutions of metal hydroxides under ambient conditions can be employed. Typically 1N-3N aqueous NaOH is employed.

The substituted unsymmetrical amino uracils can be used as intermediates for the synthesis of a variety of substituted xanthines that can fuinction as A_{2B} adeno sine receptor antagonists. Several pathways that lead to the corresponding xanthines are accessible to one skilled in the art. A typical example is shown in scheme 2.

Nitration of the amino uracil followed by reduction may afford a diamine compound which may subsequently be treated with an acid halide to form the corresponding amide as shown. This amide upon reaction under basic conditions may undergo a cyclization to generate the corresponding substituted xanthine.

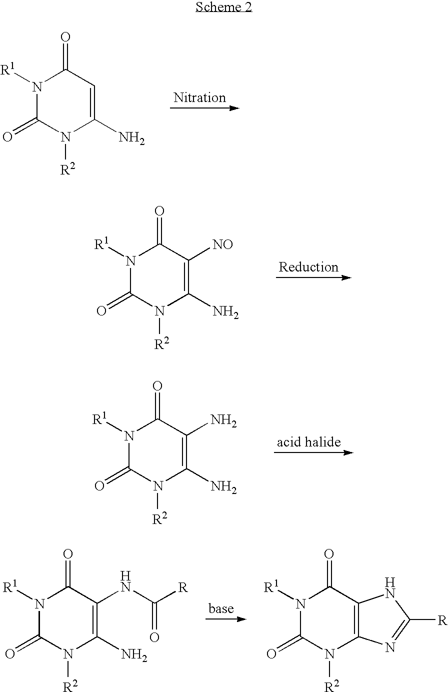

### EXAMPLES

### Experimental protocol

Compound 1. To a flask containing 2.1 g (21 mmol) of methylcyanoacetate is added 5 mL of cyclopropylamine. The mixture is allowed to stir at room temperature overnight under a nitrogen atmosphere. The remaining cyclopropylamine is removed under reduced pressure and the solid washed with diethylether to afford 2-cyano-N-cyclopropylacetamide (compound 1).

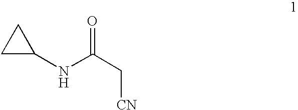

Compound 2. 2-cyano-N-cyclopropylacetamide is dissolved in 12 mL of THF in a flame-dried 50 mL round bottom flask under Nitrogen. The solution is cooled to −35° C. in a dry-ice/acetonitrile bath. NaHMDS (1.0 M in THF) is added dropwise affording complete dissolution of the compound. The mixture is allowed to stir for 15 minutes followed by the dropwise addition of phenylchloroformate to afford compound 2.

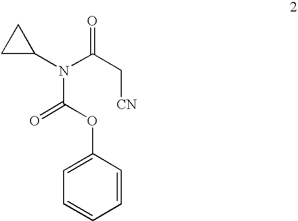

Compound 3. Treatment of the compound above with neat propylamine and heating to 50° C. for 18 h affords the target compound (3) which is further reacted without purification.

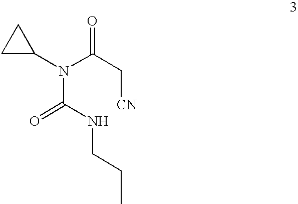

Compound 4. Compound (3) is treated with 2 N NaOH to afford the target compound (4) after stirring at room temperature overnight.

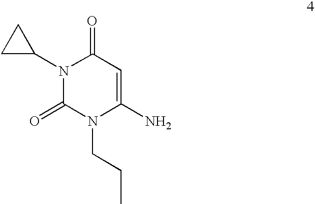

Numerous modifications and variations of the present invention are possible in light of the above teachings. It is therefore to be understood that within the scope of the appended claims, the invention may be practiced otherwise that as specifically described herein.

What is claimed is:

1. A process for preparing a compound of formula I or a pharmaceutically acceptable salt thereof, comprising: cyclizing a compound of formula II in the presence of a strong base: 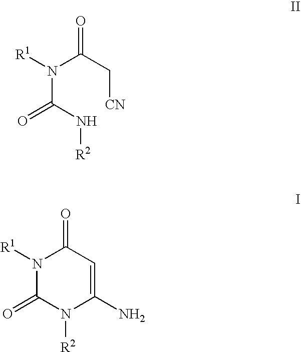
wherein:
R¹ and R² are independently hydrogen or selected from the group consisting of (C₁₋₈)alkyl, halo(C₁₋₈)alkyl, (C₃₋₈)alkenyl, (C₃₋₈)alkenyl(C₁₋₈)alkyl-, (C₃₋₈)alkynyl, (C₃₋₈)alkynyl(C₁₋₈)alkyl-, (C₁₋₈)alkoxy, (C₃₋₈)cycloalkyl, (C₃₋₈)cycloalkyl(C₁₋₈)alkyl-, (C₄₋₁₀)heterocyclyl, (C₄₋₁₀)heterocyclyl(C₁₋₈)alkyl-, (C₆₋₁₀)aryl, (C₆₋₁₀)arYloxY, (C₆₋₁₀)aryl(C₁₋₈)alkyl-, (C₅₋₁₀)heteroaryl, and (C₅₋₁₀)heteroaryl(C₁₋₈)alkyl-, wherein each group is independently substituted or unsubstituted.

2. A process for preparing a compound of formula II, comprising: 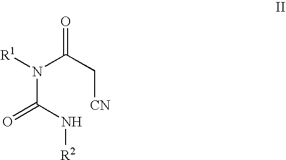
a) contacting a compound of formula III with an acylating agent of formula IV in the presence of a second strong base to produce a compound of formula V; and, 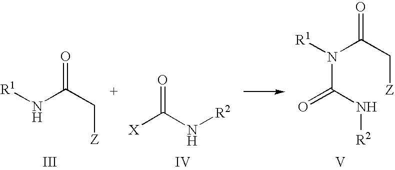
b) (i) when Z is a leaving group, contacting the compound of Formula V with M-CN to produce a compound of formula II; or,
(ii) when Z is a group that can be converted to a leaving group, converting the group to a leaving group and contacting the resulting compound with M-CN to produce a compound of formula II;
R¹ and R² are independently hydrogen or selected from the group consisting of (C₁₋₈)alkyl, halo(C₁₋₈)alkyl, (C₃₋₈)alkenyl, (C₃₋₈)alkenyl(C₁₋₈)alkyl-, (C₃₋₈)alkynyl, (C₃₋₈)alkynyl(C₁₋₈)alkyl-, (C₁₋₈)alkoxy, (C₃₋₈)cycloalkyl, (C₃₋₈)cycloalkyl(C₁₋₈)alkyl-, (C₄₋₁₀)heterocyclyl, (C₄₋₁₀)heterocyclyl(C₁₋₈)alkyl-, (C₆₋₁₀)aryl, (C₆₋₁₀)aryloxy, (C₆₋₁₀)aryl(C₁₋₈)alkyl-, (C₅₋₁₀)heteroaryl, and (C₅₋₁₀)heteroaryl(C₁₋₈)alkyl-, wherein each group is independently substituted or unsubstituted;
M is Na or K;
Z is selected from the group consisting of CN, a leaving group, or a group that can be converted to a leaving group; and, X is a leaving group.

3. A process for preparing a compound of formula II, comprising: 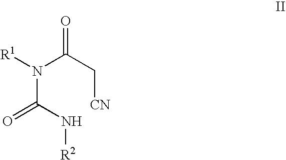
a) contacting a compound of formula III with an acylating agent of the formula VI in the presence of a third strong base to produce a compound of formula VII: 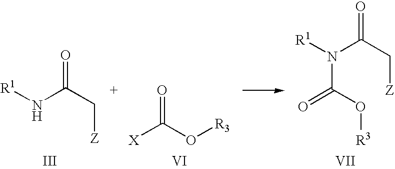
a-1) contacting the compound of formula VII with an amine of the formula R²-NH₂ to produce a compound of formula V: 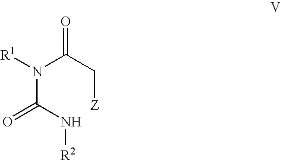
b) (i) when Z is a leaving group, contacting the compound of Formula V with M-CN to produce a compound of formula II; or,
(ii) when Z is a group that can be converted to a leaving group, converting the group to a leaving group and contacting the resulting compound with M-CN to produce a compound of formula II;
R¹ and R² are independently hydrogen or selected from the group consisting of (C₁₋₈)alkyl, halo(C₁₋₈)alkyl, (C₃₋₈)alkenyl, (C₃₋₈)alkenyl(C₁₋₈)alkyl-, (C₃₋₈)alkynyl, (C₃₋₈)alkynyl(C₁₋₈)alkyl-, (C₁₋₈)alkoxy, (C₃₋₈)cycloalkyl, (C₃₋₈)cycloalkyl(C₁₋₈)alkyl-, (C₄₋₁₀)heterocyclyl, (C₄₋₁₀)heterocyclyl(C₁₋₈)alkyl-, (C₆₋₁₀)aryl, (C₆₋₁₀)aryloxy, (C₆₋₁₀)aryl(C₁₋₈)alkyl-, (C₅₋₁₀)heteroaryl, and (C₅₋₁₀)heteroaryl(C₁₋₈)alkyl-, wherein each group is independently substituted or unsubstituted;
OR³ is a leaving group;
M is Na or K;
Z is selected from the group consisting of CN, a leaving group, or a group that can be converted to a leaving group; and,
X is a leaving group.

4. The process of claim 2, further comprising:
c) cyclizing the compound of formula II in the presence of a fourth strong base to form a compound of formula I or a pharmaceutically acceptable salt thereof:

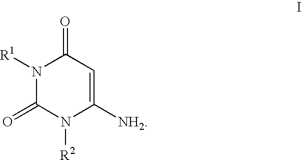

5. The process of claim 3, further comprising:
c) cyclizing the compound of formula II in the presence of a fourth strong base to form a compound of formula I or a pharmaceutically acceptable salt thereof:

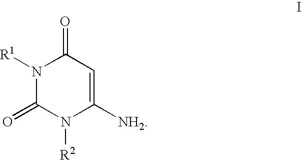

6. The process according to claim 1, wherein the strong base is a metal hydroxide.

7. The process according to claim 1, wherein the metal hydroxide base is selected from the group consisting of lithium hydroxide, calcium hydroxide, sodium hydroxide, potassium hydroxide, and mixtures thereof.

8. The process according to claim 7, wherein the metal hydroxide base is sodium hydroxide or lithium hydroxide.

9. The process according to claim 2 wherein the strong base is selected from a group consisting of n-butyl lithium, LDA, LiHMDS, NaHMDS, and KHMDS.

10. The process according to claim 9 wherein the base is NaHMDS or KHMDS.

11. The process according to claim 3 wherein the strong base is selected from a group consisting of n-butyl lithium, LDA, LiHMDS, NaHMDS, and KHMDS.

12. The process according to claim 11 wherein the base is NaHMDS or KHMDS.

13. The process according to claim 2, wherein:
R¹ is selected from a group consisting of (C₃₋₅)cycloalkyl, (C₃₋₅)cycloalkyl(C₁₋₃)alkyl-, (C₁₋₅)alkyl, and aryl(C₁₋₃)alkyl-;
R² is selected from a group consisting of (C₃₋₅)cycloalkyl, (C₃₋₅)cycloalkyl(C₁₋₃)alkyl-, (C₁₋₅)alkyl, and aryl(C₁₋₃)alkyl-; and,
X is selected from a group consisting of Cl, Br, and I.

14. The process according to claim 13, wherein:
R¹ is selected from a group consisting of ethyl, propyl, cyclopropyl, cyclobutyl, cyclopropylmethylene, cyclobutylmethylene, and benzyl;
R² is selected from a group consisting of ethyl, propyl, cyclopropyl, cyclobutyl, cyclopropylmethylene, cyclobutylmethylene, and benzyl; and,
X is selected from a group consisting of Cl, Br, and I.

15. The process according to claim 3, wherein:
R¹ is selected from a group consisting of (C₃₋₅)cycloalkyl, (C₃₋₅)cycloalkyl(C₁₋₃)alkyl-, (C₁₋₅)alkyl, and aryl(C₁₋₃)alkyl-;
R² is selected from a group consisting of (C₃₋₅)cycloalkyl, (C₃₋₅)cycloalkyl(C₁₋₃)alkyl-, (C₁₋₅)alkyl, and aryl(C₁₋₃)alkyl-;
R³ is selected from a group consisting of (C₁₋₈)alkyl, aryl(C₁₋₃)alkyl-, and aryl; and,
X is selected from a group consisting of Cl, Br, and I.

16. The process according to claim 16, wherein:
R¹ is selected from a group consisting of ethyl, propyl, cyclopropyl, cyclobutyl, cyclopropylmethylene, cyclobutylmethylene, and benzyl;
R² is selected from a group consisting of ethyl, propyl, cyclopropyl, cyclobutyl, cyclopropylmethylene, cyclobutylmethylene, and benzyl;
R³ is selected from a group consisting of methyl, ethyl, phenyl, and benzyl; and,
X is selected from a group consisting of Cl, Br, and I.

17. The process according to claim 1 wherein R¹ is cyclopropyl and R² is propyl.

18. The process according to claim 2 wherein R¹ is cyclopropyl and R² is propyl.

19. The process according to claim 3 wherein R¹ is cyclopropyl and R² is propyl.
